(19) United States
(12) Patent Application Publication
(10) Pub. No.: US 2010/0331849 A1
(43) Pub. Date: Dec. 30, 2010

(54) SPINE DISTRACTION AND COMPRESSION INSTRUMENT

(75) Inventors: RALF RIESINGER, WURMLINGEN (DE); GUNTMAR EISEN, TUTTLINGEN (DE)

Correspondence Address: Monument IP Law Group, 1717 Pennsylvania Avenue, Suite 900, Washington, DC, 20006, US

(73) Assignee: PARADIGM SPINE LLC, NEW YORK, NY (US)

(21) Appl. No.: 12/826,784

(22) Filed: Jun. 30, 2010

Related U.S. Application Data
(60) Provisional application No. 61/222,068, filed on Jun. 30, 2009.

(51) Int. Cl. A61B 17/60

(52) U.S. Cl. 606/90

(57) ABSTRACT

A surgical instrument for moving tissue, such as bone segments, and more specifically vertebrae of a spinal column, is provided. In particular, a spine compression and/or distraction instrument that can be used to reposition vertebrae is provided. Further, methods for using the surgical instrument to compress and/or distract vertebrae of a spinal column are also provided.

### CROSS-REFERENCE TO RELATED APPLICATIONS

This application claims priority to U.S. Provisional Patent Application No. 61/222,068, filed Jun. 30, 2009, and entitled “SPINE DISTRACTION AND COMPRESSION INSTRUMENT,” which is hereby incorporated by reference in its entirety.

### FIELD

The present invention relates generally to surgical instruments that are used to move tissue, and particularly bone segments, such as surgical instruments for distracting and/or compressing the spine. In particular, the present invention relates to a surgical instrument for repositioning vertebrae of a spinal column.

### BACKGROUND

The spine includes a series of joints known as motion segment units. Each unit represents the smallest component of the spine that exhibits a kinematic behavior characteristic of the entire spine. The motion segment unit is capable of flexion, extension, lateral bending, and translation. The components of each motion segment unit include two adjacent vertebrae, the corresponding apophyseal joints, an intervertebral disc, and connecting ligamentous tissue, with each component of the motion segment unit contributing to the mechanical stability of the joint. For example, the intervertebral discs that separate adjacent vertebrae provide stiffness that helps to restrain relative motion of the vertebrae in flexion, extension, axial rotation, and lateral bending.

When the components of a motion segment unit move out of position or become damaged due to trauma, mechanical injury or disease, severe pain and further destabilizing injury to other components of the spine may result. In a patient with degenerative disc disease (DDD), a damaged disc may provide inadequate stiffness, which may result in excessive relative vertebral motion when the spine is under a given load, causing pain and further damage to the disc.

Depending upon the severity of the structural changes present, currently known treatment options may include fusion, discectomy, and/or a laminectomy. Most often, these surgical treatments will also involve the use of mechanical devices such as stabilization rods or plates which are placed adjacent to the spine to secure the motion segment units in a fixed, rigid relationship. These mechanical stabilization devices can promote the natural healing of the spine in a straight spatial disposition, restore alignment to misaligned motion segment units, and enhance straightening of the spinal column in cases of disease such as scoliosis.

In some situations, the spinal rods are placed along the spinal column and various implants, such as, hooks, spacers or plates, are mounted along the rods to maintain the rods in the desired position and orientation relative to the spine. Usually, pedicle screws having rod hooks are placed onto the vertebrae, and thereafter, the rod is urged onto the hooks to straighten out the spine. In other situations, the rods can be short enough to be positioned between adjacent motion segment units using bone anchors such as pedicle screws. Here, the rod acts primarily to prevent and/or limit movement between the pairs of vertebra, thereby stabilizing these motion segment units.

Distraction and/or compression of vertebrae may be necessary prior to implantation of any spinal implant, but especially for rod-based systems. Often during the implantation process, the surgeon may need to either distract bone by pulling it away from the work site or compress bone to pull it together if broken, for example. Such would be the case where spondylolisthesis is present, a condition where adjacent vertebrae, most usually the sacrum and the lower or lumbar vertebrae, are not properly aligned or connected, such that adjacent vertebrae are displaced or the lumbar vertebrae are displaced anteriorly from the upper base of the sacrum. In a spondylolisthesis reduction, the surgeon properly repositions the vertebrae and sacrum, and then permanently joins the vertebrae and sacrum using mechanical fixation devices. The reduction may require manipulation of the vertebrae and the sacrum in one or more directions, i.e., translation in the anterior/ventral or posterior/dorsal direction, compression or distraction in the longitudinal direction of the vertebral axis, and rotation about the vertebral axis, as well as pivotal flexion of the sacrum in the ventral direction or pivotal extension of the sacrum in the dorsal direction.

The positioning of the motion segment units prior to implantation is important in order to fix the correct position of the rods and/or the implants while providing the surgeon the best visualization of the work site. It would thus be desirable to provide a surgical instrument that can either compress or distract vertebrae of a spinal column easily and effectively, while providing optimal access to the work site.

### SUMMARY

The present disclosure provides a surgical instrument for moving apart tissue, and more particularly bone segments, with respect to one another. Specifically, the surgical instrument repositions vertebrae of a spinal column by either compressing or distracting one vertebra with respect to another vertebra. The surgical instrument is configured to enable compression and/or distraction using the same instrument.

In accordance with one exemplary embodiment, a surgical instrument for moving vertebrae is provided. The instrument may include a platform comprising a pair of tracks connected together by a wall at one end and a first carrier at an opposite end, a second carrier translatable across the pair of tracks, and a pair of legs. Each of the legs is removably attachable at one end to one of the first and second carriers. At the opposite end, the legs include an anchor engaging end for engaging a bone anchor. By translating the second carrier across the platform, the user effects movement of the legs with respect to one another, and consequently, movement of the vertebrae to which the anchors are connected.

In accordance with another exemplary embodiment, a surgical instrument for repositioning tissue is provided. The instrument may include a platform comprising a pair of tracks connected together by a wall at one end and a first carrier at an opposite end, a second carrier translatable across the pair of tracks, and a pair of legs. Each of the legs is removably attachable at one end to one of the first and second carriers and includes a flattened portion for placement against tissue to be repositioned. By translating the second carrier across the platform, the user effects movement of the legs with respect to one another, and consequently urges tissue together or apart, depending on the configuration of the legs.

Also provided is a method of repositioning vertebrae of a spinal column using the disclosed surgical instrument. The method comprises providing a surgical instrument that comprises a platform including a pair of tracks connected together by a wall at one end and a first carrier at an opposite end, a second carrier translatable across the pair of tracks, and a pair of legs, each leg being removably attachable at one end to one of the first and second carriers, and having an anchor engaging end at an opposite end for engaging a bone anchor. Each leg is then attached to one of the first and second carriers, and the gripping end of each leg is placed around a bone anchor secured to a vertebra. The second carrier may be translated across the pair of tracks effects movement of one vertebra with respect to another vertebra. The translation may move the second carrier towards the first carrier, resulting in compression of the vertebrae. Alternatively, the translation may move the second carrier away from the first carrier, resulting in distraction of the vertebrae.

It is to be understood that both the foregoing general description and the following detailed description are exemplary and explanatory only and are not restrictive of the disclosure, as claimed.

Additional features of the disclosure will be set forth in part in the description which follows or may be learned by practice of the disclosure. The features of the disclosure will be realized and attained by means of the elements and combinations particularly pointed out in the appended claims.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure. In the figures:

FIG. 1A illustrates a front perspective view of a vertebral repositioning instrument configured for bone distraction in a resting position.

FIG. 1B illustrates a front perspective view of the instrument of FIG. 1A, in a deployed position.

FIG. 1C illustrates a back perspective view of FIG. 1A.

FIG. 1D illustrates a back perspective view of FIG. 1B.

FIG. 2 illustrates a front perspective view of a vertebral repositioning instrument configured for bone compression, in a resting position.

FIG. 3 illustrates an exploded view of FIG. 1A.

FIG. 4A illustrates an enlarged view of a portion of the instrument of FIGS. 1A-1D.

FIG. 4B illustrates an enlarged view of another portion of the instrument of FIGS. 1A-1D.

FIG. 4C illustrates an enlarged view of yet another portion of the instrument of FIGS. 1A-1D.

FIG. 4D illustrates an enlarged view of still yet another portion of the instrument of FIGS. 1A-1D.

FIG. 5A illustrates a perspective view of the instrument of FIGS. 1A-1D engaged with bone screws.

FIG. 5B illustrates a perspective view of the instrument of FIG. 2 engaged with some exemplary bone screws.

FIG. 6A illustrates a perspective view of another exemplary embodiment of a repositioning instrument of the present disclosure.

FIGS. 6B and 6C illustrate the steps of engaging the repositioning instrument of FIG. 6A with some exemplary bone screws.

FIG. 7A illustrates a perspective view of yet another exemplary embodiment of a repositioning instrument of the present disclosure.

FIGS. 7B and 7C illustrate the steps of distracting tissue with the repositioning instrument of FIG. 7A.

### DESCRIPTION OF THE EMBODIMENTS

The present disclosure provides a surgical instrument for moving bone segments, and more specifically, vertebrae of a spinal column. In particular, the present disclosure provides a spine compression and/or distraction instrument that can be used to reposition vertebrae. In some embodiments, the instrument is configurable to allow for switching between compression or distraction based on interchangeable components. The present disclosure further provides methods for using the surgical instrument to compress and/or distract vertebrae of a spinal column. In addition, various embodiments of the instrument may be configured to compress or distract during surgery.

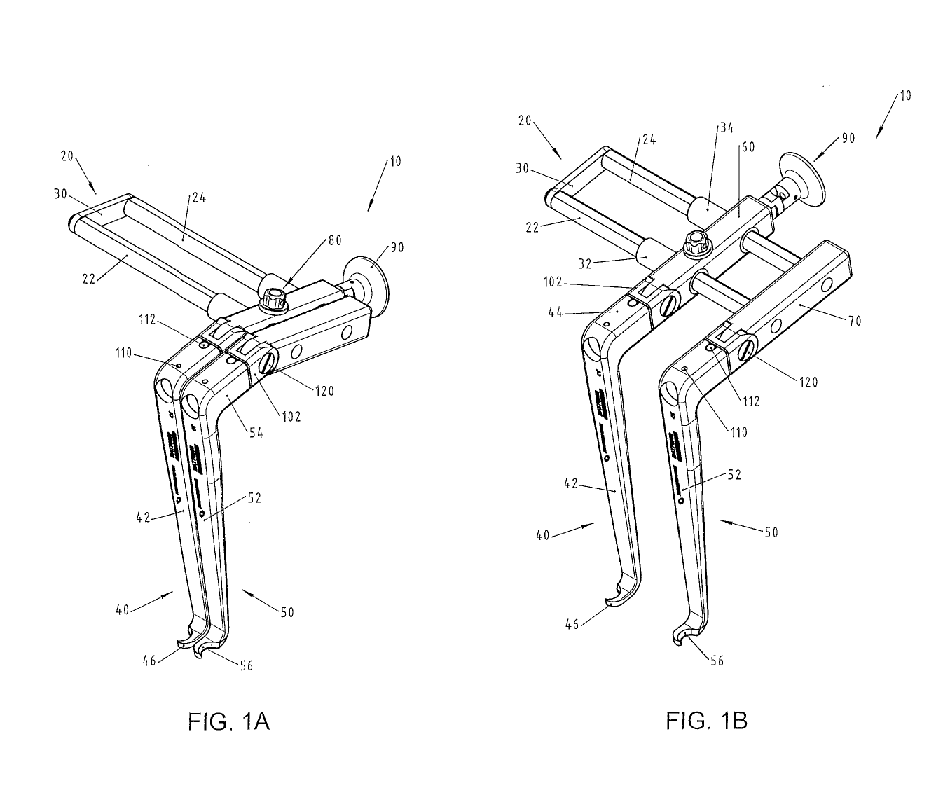

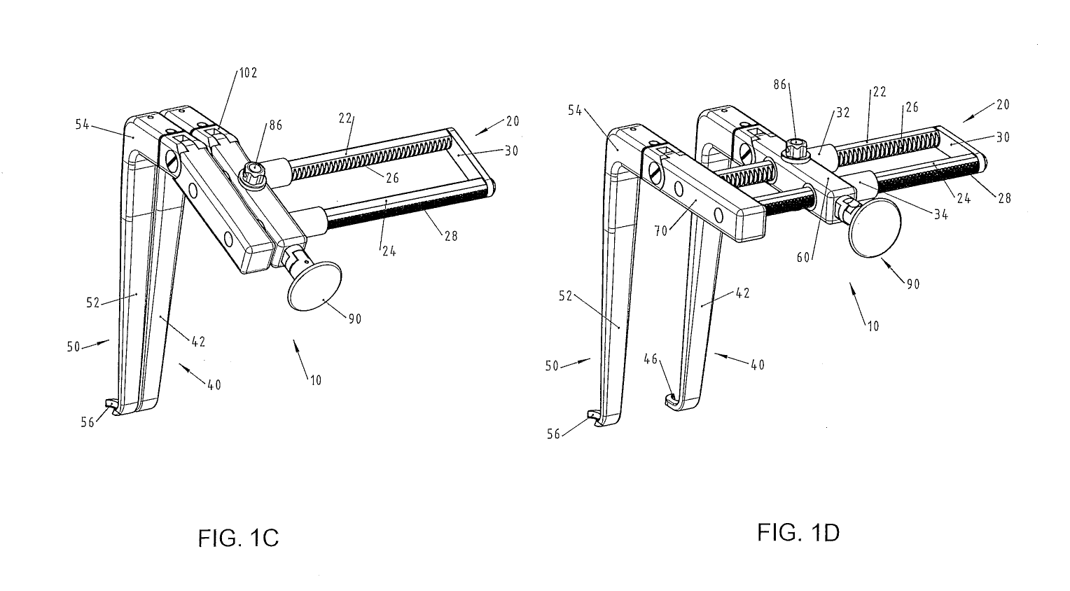

FIGS. 1A-1D show a surgical instrument 10 according to one embodiment. As shown, the surgical instrument 10 may comprise a platform having tracks along which one or more carriers may move. The carriers provide mechanical reach to the desired site and also provide structure for the desired movement of the bone segment and/or tissue. During use, various interchangeable parts may be fitted to the carriers of the surgical instrument to configure it for compression or distraction, as well as coupling the instrument to a bone segment or bone anchoring device. In the embodiment shown in FIGS. 1A-1D, the surgical instrument 10 is shown having substantially linear tracks or rails along which its carriers can translate. Other embodiments may employ different mechanisms for allowing translation of the carriers, such as a screw type shaft, a scissor or cross member construction, and the like.

Referring now to FIGS. 1A-1D, the surgical instrument 10 includes a platform 20 that supports a pair of carriers 60, 70. The platform 20 comprises a first track 22 and second track 24 joined together at connector wall 30. Fixed carrier 70 is rigidly secured to first and second tracks 22, 24, whereas movable carrier 60 can translate across the first and second tracks 22, 24. Collectively, the first and second tracks 22, 24, the connector wall 30, and the fixed carrier 70 cooperate together to form the rigid mechanical framework of the platform 20 onto which the movable carrier 60 can slide across. Both a translation unit 80 and a locking unit 90 are provided with movable carrier 60 to control the amount of its translation, or movement, across the platform 20.

Each of carriers 60, 70 is attached to a hinged arm 100 that is configured to allow pivoting and interchangeable connectivity with one of legs 40, 50. As shown, each one of legs 40, 50 comprises an L-shaped shaft portion 42, 52 which terminates at one end into a gripping end 46, 56 configured as, for example, a hook or claw, for engaging a bone anchor or screw 200. The shaft portion 42, 52 terminates at an opposite end at a connecting end 44, 54 configured for interchangeable attachment with the hinged arm 100.

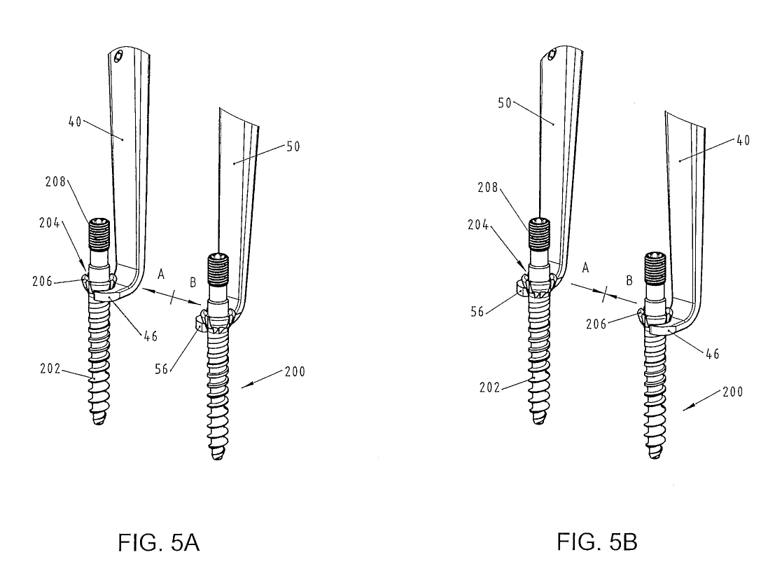

For purposes of illustration, FIGS. 1A-1D represent the instrument 10 in the distraction mode, whereby translation of the movable carrier 60 relative to the fixed carrier 70 enables a surgeon to push apart a pair of bone screws 200 (not shown in FIGS. 1A-1D) secured to the spine and consequently distract the vertebrae themselves. More specifically, FIG. 1A shows the instrument 10 in a resting state with legs 40, 50 connected such that the gripping ends 46, 56 face away from one another (i.e., the hooks turn away from one another). The legs 40, 50 are additionally shown pivoted relative to the carriers 60, 70. In contrast, FIG. 1B shows the same instrument 10 in an active state whereby the movable carrier 60 is displaced relative to the fixed carrier 70 and the gripping ends 46, 56 have been moved apart. Another illustration of how the surgical instrument 10 may be coupled to a bone screw 200 for distraction is provided with reference to FIG. 5A below.

FIGS. 1C and 1D show back views of instrument 10, with FIG. 1C corresponding to the back view of FIG. 1A and FIG. 1D corresponding to the back view of FIG. 1B. FIG. 1C shows the instrument 10 in the distraction mode, at rest, with the locking unit 90 in a locked position such that the movable carrier 60 is prevented from translating backwards on the platform 20. In the locked position, the locking unit 90 engages one of the grooves or indentations 28 that span across the length of the second track 24, preventing movement of the movable carrier 60 backwards across the platform 20. Turning the knob 86 of the translation unit 80 effects controlled, stepwise or ratcheting movement of the carrier 60 forward across the row of teeth 26 along the first track 22.

In contrast, FIG. 1D shows the instrument 10 in the active state, with the locking unit 90 in the unlocked position. In the unlocked position, the locking cap 96 is rotated 90 degrees from the locked position, and the locking unit 90 disengages from any one of the grooves or indentations 28 spanning across the second track 24 thereby enabling the movable carrier 60 to freely slide across the second track 24. In this position, it is possible to move the carrier 60, for example, by manually pushing on it.

The combination of locking unit 90 and translation unit 80 on the movable carrier 60 provides the instrument 10 with simple, effective and easy to use mechanisms for controlling and regulating the amount of movement that can be achieved. Further, as previously described, the legs 40, 50 are interchangeable and can be attached to either the movable carrier 60 or the fixed carrier 70.

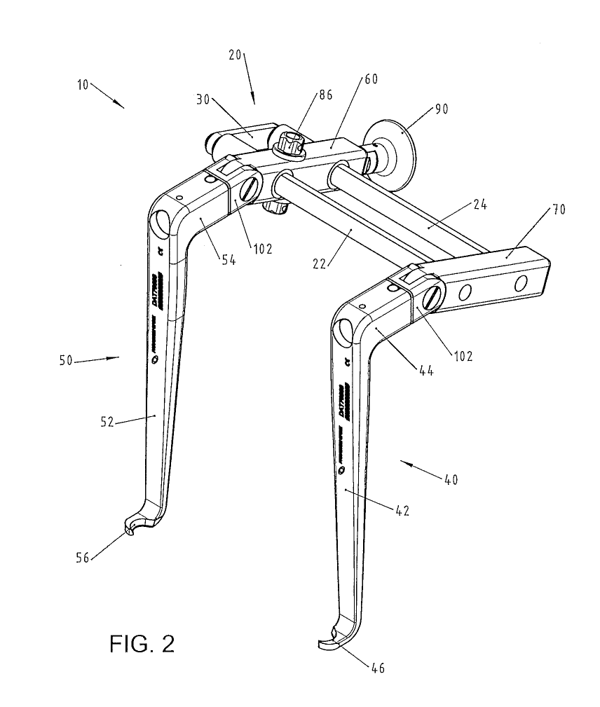

Referring now to FIG. 2, the instrument 10 may be readily configured for compression based on interchanging some of its components. For example, FIG. 2 shows the instrument 10 in a resting state, with leg 40 attached to fixed carrier 70 and leg 50 attached to movable carrier 60. In this configuration, the instrument 10 is in its compression mode, with the hooks of the gripping ends 46, 56 facing towards one another. Another illustration of how the surgical instrument 10 may be coupled to a bone screw 200 for compression is provided with reference to FIG. 5B below.

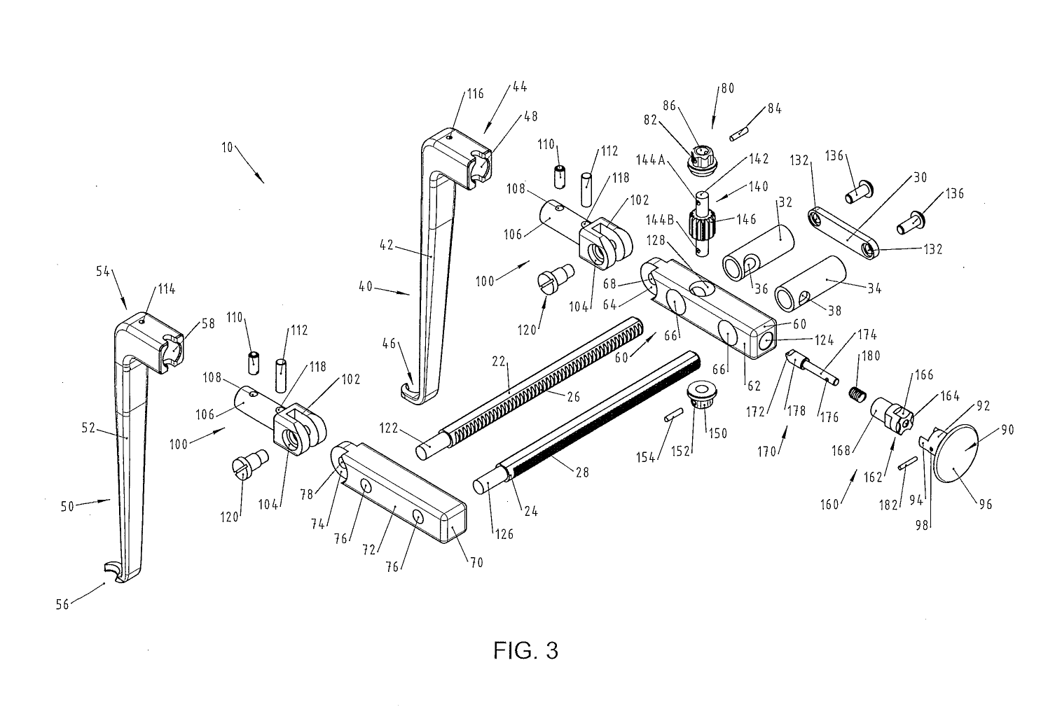

Turning now to FIG. 3, an exploded view is provided to illustrate some further details of the instrument 10. To assemble instrument 10, the head 122 of first track 22 and the head 126 of second track 24 can be securely connected to the body 72 of fixed carrier 70 through track holes 76. The movable carrier 60 can include a corresponding pair of track holes 66 on the carrier body 62 for placement of the first and second tracks 22, 24 therethrough.

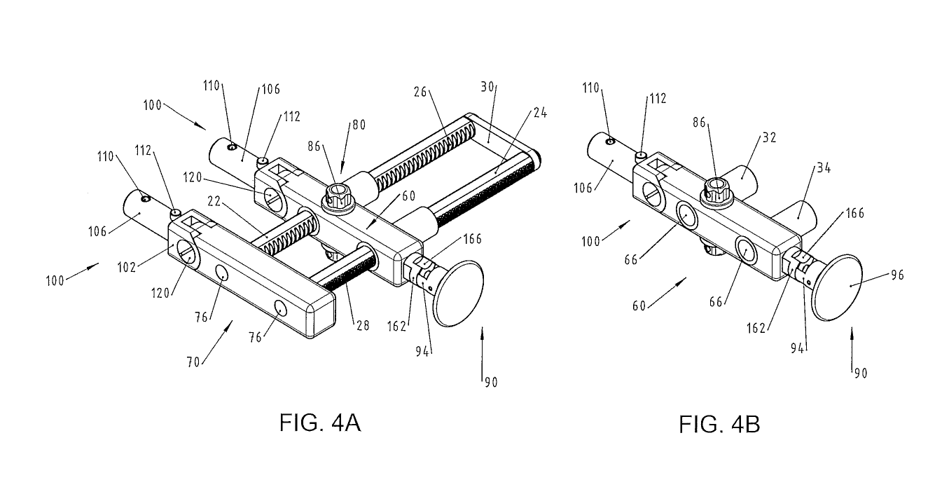

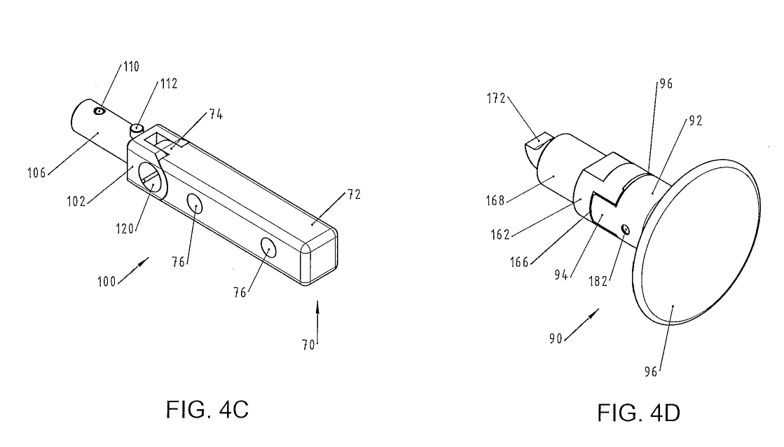

A pair of sleeves 32, 34 can also be provided with movable carrier 60 to serve as additional support for the tracks 22, 24. First and second sleeves 32, 34 can be configured as hollow cylinders to allow the first and second tracks 22, 24 to pass through. As further described below, FIG. 4A provides another illustration of this portion of the surgical instrument 10. Connector wall 30 can include a pair of screw holes 132 for securing screws 136 to the first and second tracks 22, 24.

Movable carrier 60 includes a translation unit opening 128 for the translation unit 80. This translation unit 80 comprises a ratcheting pin 140 having a shaft 142 around which there is a belt of teeth 146. At both ends of the shaft 142 are pin holes 144A, 144B. The ratcheting pin 140 resides within the opening 128 of the movable carrier 60. First sleeve 32 is provided with a cutaway portion 36 to accommodate the belt of teeth 146, which engages with the ratcheting teeth 36 of first track 22 in use. To keep the ratcheting pin 140 secured in place, a cap 150 with a pin hole 152 can be provided below the movable carrier 60. Pin 154 can then be placed through pin hole 152 and pin hole 144B to secure the ratcheting pin 140 below the carrier body 62. Knob 86 with pin hole 82 can also be provided along with pin 84 that can be placed through pin hole 144A of the ratcheting pin 140 to secure the ratcheting pin 140 above the carrier body 62. Turning the knob 86 will cause the ratcheting pin 140 to move along the ratcheting teeth 26 of first track, thereby allowing the user to move the carrier 60 in controlled incremental steps.

Carrier body 62 also includes a locking unit opening 124 on one of its ends for accommodating the locking unit 90. As shown, locking unit 90 can comprise a locking cap 96 attached to a stem 92 having diametrically opposed tabs 94 extending therefrom. Also provided is a plunger 170 having a shaft 174 with a pin hole 176, the shaft 174 extending into a widened portion 178 that ends at a bevel edged tip 172. The plunger 170 is secured to locking cap 96 by pin 182 which extends through holes 176 and 98. A spring 180 can be positioned between the plunger 170 and the locking cap 96 to provide a biasing force against the locking cap 96.

As further shown in FIG. 3, a hub 160 is provided which includes a cylindrical body 162 attached to a stem 168. The body 162 includes a pair of diametrically opposed cutaway portions 166 that correspond to the tabs 94 of the locking cap 96. An opening 164 down the midline enables the shaft 174 of the plunger 170 to extend through the hub 160.

The locking unit 90 can be assembled by placing the spring 180 inside the hub 160, and inserting the shaft 174 of the plunger 170 through the opening 164 of the hub 160. Next, the locking cap 96 is secured to the plunger 170 by placing pin 182 through openings 176 and 98 on the plunger 170 and the locking cap 96, respectively. The hub 160 can then be placed inside locking unit opening 124 of the movable carrier body 62. The locking unit 90 should be positioned such that the bevel edged tip 172 of the plunger 170 can engage the row of grooves or indents 28 of the second track 24. Once the locking unit 90 is properly aligned, the hub 160 can be secured in place with, for example, an adhesive. A complete locking unit 90 is also shown with reference to FIG. 4D.

In use, a surgeon may rotate the locking cap 96 such that the tabs 94 reside within the cutaway portions 166 (as also shown in FIG. 4D) to put the instrument 10 in the locked position. The bevel edged tip 172 can engage one of the grooves 28 of second track 24. This prevents translation of the movable carrier 60 backwards on the platform 20.

When translation is desired, the surgeon may rotate the knob 86 to effect a stepwise, incremental movement of the carrier 60 forward. When the instrument 10 is in compression mode, locking the instrument 10 prevents distraction. When the instrument 10 is in distraction mode, locking the instrument 10 prevents compression.

To unlock the cap 96, the locking cap 96 may be turned 90 degrees such that the tabs 94 rest on top of the hub body 162 (as also shown in FIGS. 4A and 4B). In this unlocked position, the bevel edged tip 172 is pulled away from the row of grooves 28 and the surgeon can freely move the carrier 60 across the platform 20 as needed.

Both movable carrier 60 and fixed carrier 70 can include a tabbed end 64, 74 having a hole therethrough 68, 78. In the embodiment shown, the tabbed end 64, 74 fits within U-shaped head 102 of hinged arm 100. The head 102 attaches to carrier 60, 70 with a bolt 120 that extends through hole 104 and hole 68, 78. This head 102 extends into a stem 106 on which resides two unique and distinct pegs (as also shown in FIGS. 4A-4C).

The first peg 110 resides in first hole 108 and is configured as a spring peg. The first peg 110 is spring deployable and engages opening 114, 116 of legs 50, 40. The second peg 112 resides in second hole 118 of the stem 106 and acts as an anti-rotation mechanism. The second peg 112 engages with the notched opening 48, 58 of leg 40, 50.

As previously described, the legs 40, 50 are interchangeable and can be attached to the hinged arm 100 of either the movable carrier 60 or the fixed carrier 70. By pulling on the legs 40, 50, the user can dislodge the spring peg 110 from opening 116, 114 and thereby loosen the arm 50, 40 off the hinged arm 100. Attachment of the legs 40, 50 is similarly easy. The user slides the notched opening 48, 58 of the leg 40, 50 over the first and second pegs 110, 112 until the first peg 110 engages opening 116, 114 and the notch of the opening 48, 58 engages the second peg 112. Since hinged arm 100 is freely pivotable with respect to the carriers 60, 70, the legs 40, 50 are able to also pivot with respect to the carriers 60, 70. If desired, a mechanism may be provided to enable the user to control the amount of pivoting between the legs 40, 50 and the carriers 60, 70.

Providing the instrument user with the ability to readily interchange the position of the legs 40, 50 allows the surgeon to adapt the instrument 10 to the patient's anatomy or needs quickly and easily.

During surgery, the user can determine whether the instrument 10 is to be used for compression or distraction. The user can then configure the instrument 10 for compression mode or distraction mode by attaching each leg 40, 50 to one of the first and second carriers for that desired configuration. After the instrument 10 has been configured to the appropriate mode, the gripping ends 46, 56 of the legs 40, 50 are placed around a bone anchor, usually a bone screw, secured to a vertebra. The user can then effect movement of the vertebrae by translating the second carrier 60 across the pair of tracks 22, 24, which results in movement of one vertebra with respect to another vertebra.

In general, movement of the second carrier 60 towards the first carrier 70 will typically result in compression of the bone screws 200 and attached vertebrae (not shown). Conversely, moving the second carrier 60 away from the first carrier 70 typically results in distraction of the bone screws 200 and attached vertebrae (not shown). Since the legs 40, 50 are freely pivotable, the user can easily manipulate the instrument 10 relative to the patient's anatomy in the direction and orientation desired, while still being able to effectively reconfigure and realign the bone segments in an atraumatic manner.

Referring now to FIG. 5A, if distraction is desired, the legs 40, 50 of the surgical instrument 10 are attached such that their gripping ends 46, 56 face away from one another. During use, the displacement of movable carrier 60 along direction A-B can thus effect the movement of the bone screw 200 onto which the gripping end 46 is attached and thereby distract this attached vertebra relative to the vertebra (by way of bone screw 200) on which gripping end 56 is mounted. As shown in FIG. 5A, each screw 200 may typically comprise a threaded shaft 202 extending into a flange or shoulder 206 at the junction where the screw head 204 extends. The screw head 204 can terminate into a threaded section 208 for attachment to other fasteners, implants or rod-based systems as needed. The flange 206 sits above the outer surface of the bone segment, providing a suitable structure for engaging the gripping ends 46, 56 of legs 40, 50 in an atraumatic manner. As the user moves leg 40 along direction A-B, the bone screws 200 become distracted and consequently the vertebrae (not shown) to which the bone screws 200 are attached are thereby distracted.

Referring now to FIG. 5B, if compression by the surgical instrument 10 is desired, the legs 40, 50 are attached to the surgical instrument 10 such that their gripping ends 46, 56 are facing one another. As shown, the instrument 10 is engaged with a pair of bone screws 200. As the user moves leg 40 along direction A-B, the bone screws 200 become compressed and consequently the vertebrae (not shown) to which the bone screws 200 are attached are thereby compressed.

While the present embodiments have been described with pins, it is contemplated that any comparable mechanical fastening device, such as for example, screws, bolts, rivets, etc. can be substituted without departing from the spirit of the invention. Likewise, while the present embodiments are described with adhesive, other suitable mechanisms for securing the structural elements together can be utilized, such as for example, soldering, welding, or creating an interference fit between elements.

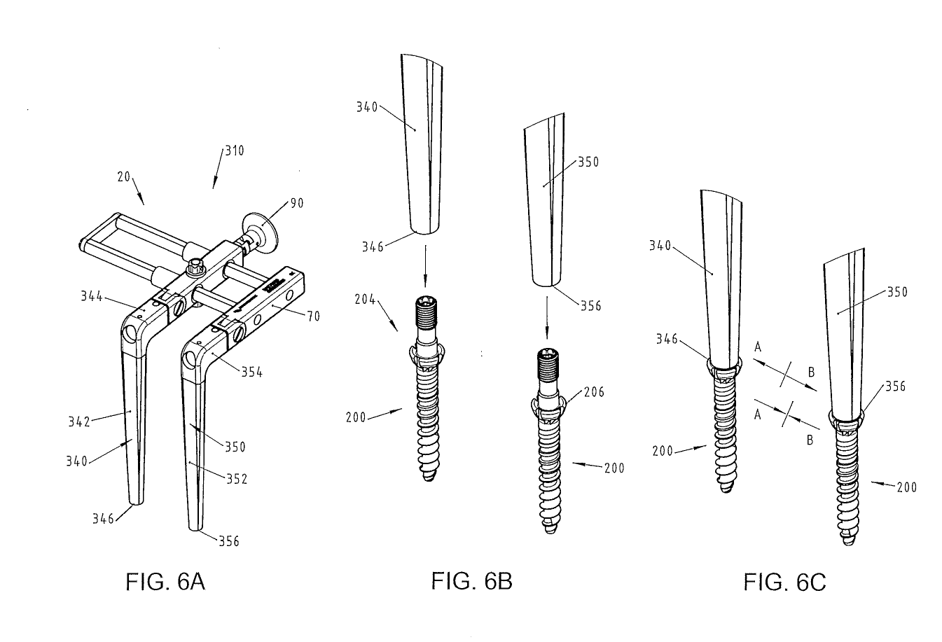

FIG. 6A illustrates another exemplary embodiment of a surgical repositioning instrument 310 of the present disclosure. Surgical instrument 310 shares several common features of the surgical instrument 10 described above, with like elements having the same numerals, except that surgical instrument 310 includes open-ended tubular legs 340, 350 instead of gripping-ended legs 40, 50. Like legs 40, 50, each one of open-ended legs 340, 350 comprises an L-shaped shaft portion 342, 352 which terminates into an open, screw-receiving tubular end 346, 356 configured to receive and engage a portion of a bone anchor or screw 200 such as the ones described above for FIGS. 5A and 5B. The shaft portion 342, 352 terminates at an opposite end at a connecting end 344, 354 configured for interchangeable attachment with the hinged arm 100 of carriers 60, 70, similar to gripping-ended legs 40, 50.

Referring now to FIGS. 6B and 6C, in use, the open-ended legs 340, 350 may be positioned over bone anchors or screws 200. The legs 340, 350 may be slid onto the screws 200 until the ends 346, 356 rest against the shoulder or flange 206 portion of the screw head 204. Once engaged, the user is then able to maneuver the bone anchors 200 along the directions indicated by arrowed lines A-B by moving legs 340, 350 with respect to one another to either compress or distract as previously described for surgical instrument 10, and thereby effect the repositioning of vertebrae attached to the bone anchors 200.

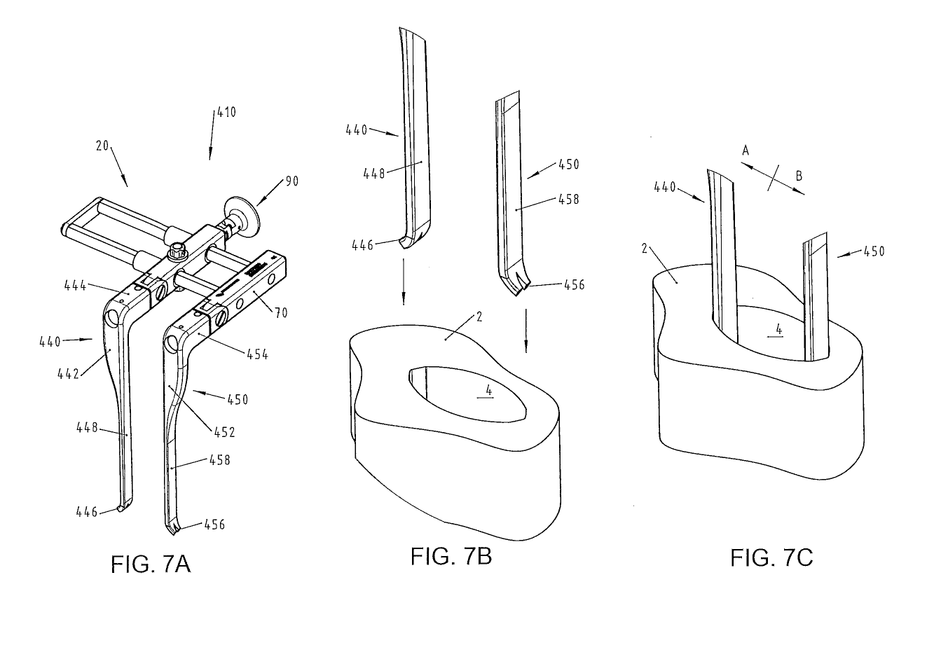

FIG. 7A illustrates yet another exemplary embodiment of a surgical instrument 410 of the present disclosure that can be used to reposition (distract or compress) tissue during surgery. Surgical instrument 410 shares several common features of surgical instrument 10 described above, with like elements having the same numerals, except that surgical instrument 410 includes tissue-moving legs 440, 450 instead of gripping-ended legs 40, 50. Like legs 40, 50, each one of tissue-moving legs 440, 450 comprises an L-shaped shaft portions 442, 452 that terminate into a tissue-gripping end 446, 456 configured to engage tissue 2 adjacent an opening 4. The shaft portion 442, 452 also includes a flattened portion 448, 458 for placement against tissue 2 to be moved, and terminates at an opposite end at a connecting end 444, 454 configured for interchangeable attachment with the hinged arm 100 of carriers 60, 70, similar to gripping-ended legs 40, 50.

Referring now to FIGS. 7B and 7C, in the configuration shown, the tissue-moving legs 440, 450 may be positioned for insertion into a slot or opening 4 formed in tissue 2 to widen the opening. The legs 440, 450 may be slid down so that the flattened portions 448, 458 of the legs 440, 450 rest against the tissue 2 surrounding the opening 4, and the tissue-gripping ends 446, 456 engage the tissue 2. Once engaged, the user is then able to widen the opening 4, moving apart the surrounding tissue 2 as desired, by moving apart legs 440, 450 along the direction indicated by arrowed line A-B as previously described for surgical instrument 10.

Although FIGS. 7A and 7B show the surgical instrument 410 configured for distraction, it is understood that the surgical instrument 410 may easily be configured for compression by switching the position of legs 440, 450. In this scenario, the tissue-gripping ends 446, 456 would face toward one another and allow the flattened portions 448, 458 to compress tissue rather than distract.

Other embodiments of the invention will be apparent to those skilled in the art from consideration of the specification and practice of the disclosure provided herein. It is intended that the specification and examples be considered as exemplary only, with a true scope and spirit of the disclosure being indicated by the following claims.

1. A surgical instrument for moving vertebrae, comprising:
a platform including a pair of tracks connected together by a wall at one end and a first carrier at an opposite end;
a second carrier translatable across the pair of tracks;
a pair of legs, each leg being removably attachable at one end to one of the first and second carriers, and having an anchor engaging end at an opposite end for engaging a bone anchor; and
wherein translation of the second carrier effects movement of one vertebra with respect to another vertebra.

2. The instrument of claim 1, wherein the first carrier is rigidly fixed to the pair of tracks.

3. The instrument of claim 1, wherein the anchor engaging end is configured as a hook.

4. The instrument of claim 1, wherein the anchor engaging end is configured as an open end.

5. The instrument of claim 1, further including a translating unit for controlling the movement of the second carrier.

6. The instrument of claim 1, further including a locking unit for controlling the movement of the second carrier.

7. The instrument of claim 1, wherein the legs are pivotable with respect to the carriers.

8. The instrument of claim 1, wherein each leg is L-shaped.

9. A surgical instrument for repositioning tissue, comprising:
a platform including a pair of tracks connected together by a wall at one end and a first carrier at an opposite end;
a second carrier translatable across the pair of tracks;
a pair of legs, each leg being removably attachable at one end to one of the first and second carriers, and having a flattened portion for placement against tissue; and
wherein translation of the second carrier effects movement of tissue.

10. The instrument of claim 8, wherein each leg further includes a tissue-gripping end.

11. The instrument of claim 8, wherein each leg is L-shaped.

12. A method for moving vertebrae, comprising the steps of:
providing a surgical instrument including a platform including a pair of tracks connected together by a wall at one end and a first carrier at an opposite end, a second carrier translatable across the pair of tracks, and a pair of legs, each leg being removably attachable at one end to one of the first and second carriers, and having an anchor engaging end at an opposite end for engaging a bone anchor;
attaching each leg to one of the first and second carriers;
engaging the anchor engaging end of each leg with a bone anchor secured to a vertebra; and
translating the second carrier across the pair of tracks.

13. The method of claim 12, wherein the anchor engaging end comprises a hook, and the step of engaging comprises placing the hook around the bone anchor.

14. The method of claim 12, wherein the anchor engaging end comprises an open end, and the step of engaging comprises placing the open end over a portion of the bone anchor.

15. The method of claim 12, wherein the step of translating effects movement of one vertebra with respect to another vertebra.

15. The method of claim 12, wherein the step of translating comprises moving the second carrier towards the first carrier.

16. The method of claim 15, wherein the step of moving the second carrier results in compression of the vertebrae.

17. The method of claim 12, wherein the step of translating comprises moving the second carrier away from the first carrier.

18. The method of claim 17, wherein the step of moving the second carrier results in distraction of the vertebrae.
